(19) United States
(12) Patent Application Publication
(10) Pub. No.: US 2019/0033505 A1
(43) Pub. Date: Jan. 31, 2019

(54) EAR-WORN ELECTRONIC DEVICE WAVEGUIDE EXTENSION FOR INNER EAR WAVEFORM TRANSMISSION

(71) Applicant: Starkey Laboratories, Inc., Eden Prairie, MN (US)

(72) Inventors: Peggi S. Cross, Tucson, AZ (US); Kaysar Rahim, Cohoes, NY (US)

(21) Appl. No.: 16/028,015

(22) Filed: Jul. 5, 2018

Related U.S. Application Data
(60) Provisional application No. 62/537,087, filed on Jul. 26, 2017.

(51) Int. Cl. F21V 8/00; A61B 5/00; H04R 1/10

(52) U.S. Cl. CPC G02B 6/0001; A61B 5/742; H04R 1/105; A61B 5/6817; A61B 5/0082

(57) ABSTRACT

An ear-worn electronic device comprises a housing configured for insertion into an ear canal and comprising a proximal section and a distal section. The distal section comprises a distal end configured to terminate prior to a second bend of the ear canal when the housing is fully inserted into the ear canal. An inner waveguide is disposed in the housing and extends to the distal end. A waveguide extension is coupled to the distal end and dimensioned to extend past the second bend of the ear canal when the housing is fully inserted into the ear canal. The waveguide extension is communicatively coupled to the inner waveguide. An articulation mechanism is situated between the waveguide and the waveguide extension. The articulation mechanism is configured to facilitate articulation of the waveguide extension relative to the inner waveguide during insertion of the housing into the ear canal.

### RELATED PATENT DOCUMENTS

This application claims the benefit of Provisional Patent Application Ser. No. 62/537,087 filed on Jul. 26, 2017, to which priority is claimed pursuant to 35 U.S.C. § 119(e), and which is incorporated herein by reference in its entirety.

### TECHNICAL FIELD

This application relates generally to hearing devices, including ear-worn electronic devices, hearing aids, personal amplification devices, and other hearables.

### SUMMARY

Various embodiments are directed to an ear-worn electronic device comprising a housing configured for insertion into an ear canal. The housing comprises a proximal section and a distal section. The distal section comprises a distal end configured to terminate prior to a second bend of the ear canal when the housing is fully inserted into the ear canal. An inner waveguide is disposed in the housing and extends to the distal end. A waveguide extension is coupled to the distal end and dimensioned to extend past the second bend of the ear canal when the housing is fully inserted into the ear canal. The waveguide extension is communicatively coupled to the inner waveguide. An articulation mechanism is situated between the waveguide and the waveguide extension. The articulation mechanism is configured to facilitate articulation of the waveguide extension relative to the inner waveguide during insertion of the housing into the ear canal.

According to other embodiments, an ear-worn electronic device comprises a housing configured for insertion into an ear canal. The housing comprises a proximal section and a distal section. The distal section comprises a distal end configured to terminate prior to a second bend of the ear canal when the housing is fully inserted into the ear canal. An inner waveguide is disposed in the housing and extends to the distal end. A waveguide extension is coupled to the distal end and dimensioned to extend past the second bend of the ear canal when the housing is fully inserted into the ear canal. The waveguide extension is communicatively coupled to the inner waveguide. An articulation mechanism is situated between the waveguide and the waveguide extension. The articulation mechanism is configured to facilitate articulation of the waveguide extension relative to the inner waveguide during insertion of the housing into the ear canal. An infrared sensor is disposed in the housing and communicatively coupled to the inner waveguide. A processor is disposed in the housing and coupled to the infrared sensor. The processor is configured to measure a physiologic signal or condition in response to a waveform received by the infrared sensor.

The above summary is not intended to describe each disclosed embodiment or every implementation of the present disclosure. The figures and the detailed description below more particularly exemplify illustrative embodiments.

### BRIEF DESCRIPTION OF THE DRAWINGS

Throughout the specification reference is made to the appended drawings wherein:

FIG. 1A is an illustration of a person's inner ear including various anatomical features;

FIG. 1B is an illustration of an ear-worn electronic device having an inner waveguide and a waveguide extension in accordance with various embodiments;

FIG. 2 is an illustration of an ear-worn electronic device having a detachable waveguide extension in accordance with various embodiments;

FIG. 3A illustrates an ear-worn electronic device having an inner waveguide and a waveguide extension in accordance with various embodiments;

FIG. 3B shows details of the inner waveguide and the waveguide extension shown in FIG. 3A; and

FIG. 4 illustrates an ear-worn electronic device having an inner waveguide and a waveguide extension in accordance with various embodiments; and

FIG. 5 is a block diagram showing various components of an ear-worn electronic device that can be configured to incorporate a waveguide comprising an inner waveguide and a waveguide extension in accordance with various embodiments.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

It is understood that the embodiments described herein may be used with any ear-worn electronic device without departing from the scope of this disclosure. The devices depicted in the figures are intended to demonstrate the subject matter, but not in a limited, exhaustive, or exclusive sense. Ear-worn electronic devices, such as hearables (e.g., wearable earphones and earbuds), hearing aids, and hearing assistance devices, typically include an enclosure, such as a housing or shell, within which internal components are disposed. Typical components of an ear-worn electronic device can include a digital signal processor (DSP), memory, power management circuitry, one or more communication devices (e.g., a radio, a near-field magnetic induction (NFMI) device), one or more antennas, one or more microphones, and a receiver/speaker, for example. Some ear-worn electronic devices can incorporate a long-range communication device, such as a Bluetooth® transceiver or other type of radio frequency (RF) transceiver. A communication device (e.g., a radio or NFMI device) of an ear-worn electronic device can be configured to facilitate communication between a left ear device and a right ear device of the ear-worn electronic device.

Ear-worn electronic devices of the present disclosure can incorporate an antenna arrangement coupled to a high-frequency radio, such as a 2.4 GHz radio. The radio can conform to an IEEE 802.11 (e.g., WiFi®) or Bluetooth® (e.g., BLE, Bluetooth® 4.2 or 5.0) specification, for example. It is understood that hearing devices of the present disclosure can employ other radios, such as a 900 MHz radio. Ear-worn electronic devices of the present disclosure can be configured to receive streaming audio (e.g., digital audio data or files) from an electronic or digital source. Representative electronic/digital sources (e.g., accessory devices) include an assistive listening system, a TV streamer, a radio, a smartphone, a laptop, a cell phone/entertainment device (CPED) or other electronic device that serves as a source of digital audio data or other types of data files. Ear-worn electronic devices of the present disclosure can be configured to effect bi-directional communication (e.g., wireless communication) of data with an external source, such as a remote server via the Internet or other communication infrastructure.

The term ear-worn electronic device of the present disclosure refers to a wide variety of ear-level electronic devices that can aid a person with impaired hearing. The term ear-worn electronic device also refers to a wide variety of devices that can produce optimized or processed sound for persons with normal hearing. Ear-worn electronic devices of the present disclosure include hearables (e.g., wearable earphones, headphones, earbuds, virtual reality headsets), and hearing aids (e.g., hearing instruments), for example. Ear-worn electronic devices include, but are not limited to in-the-ear (ITE), in-the-canal (ITC), invisible-in-canal (IIC) or completely-in-the-canal (CIC) type hearing devices or some combination of the above. Throughout this disclosure, reference is made to an “ear-worn electronic device,” which is understood to refer to a system comprising a single ear device (left or right) or both a left ear device and a right ear device.

There is a need to transmit light from the inner ear to an optical sensor placed inside an ear-worn electronic device configured for insertion in a person's ear. There is a particular need to transmit light from the inner ear to an optical sensor placed inside the shell of a hearing device, such as an in-the-canal (ITC) or in-the-ear (ITE) hearing aid, using a waveguide. Some applications require the waveguide to extend past the second bend of the ear canal in order to gather light from the innermost canal or the tympanic membrane (ear drum). A fixed structure that extends past the second bend of the canal and has the contour of the ear canal cannot be easily or painlessly inserted into the ear.

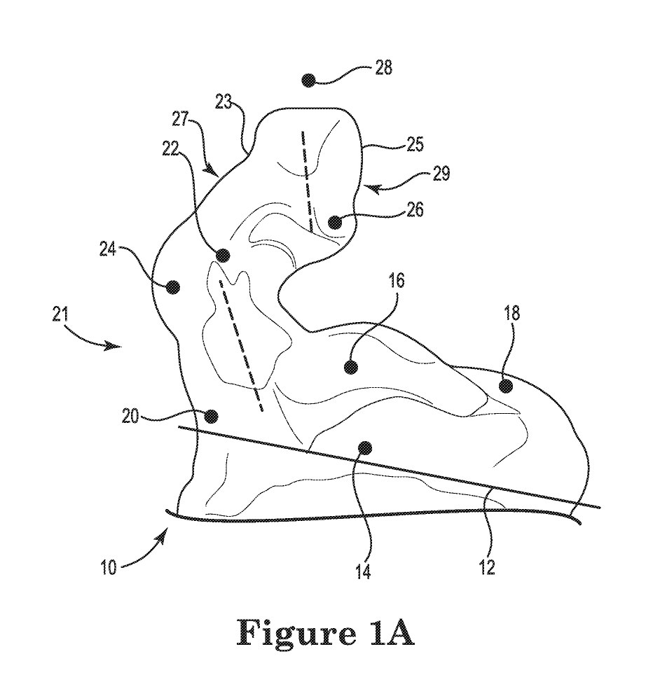

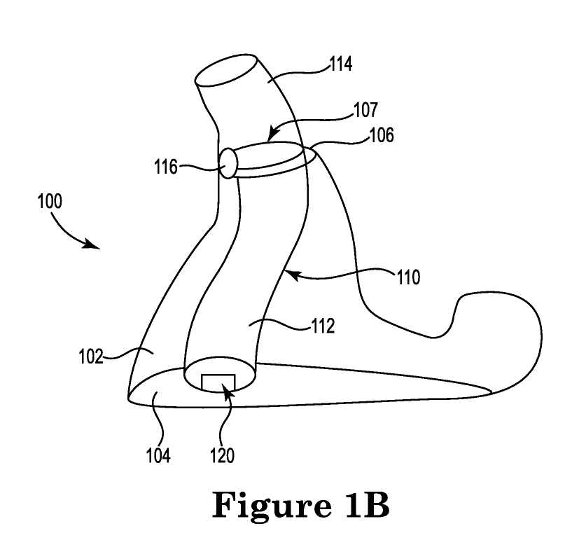

FIG. 1A is an illustration of a person's inner ear 10 and, in particular, the ear canal 22. The inner ear 10 illustrated in FIG. 1A shows a number of anatomical features near the earline 12, including the antitragus 14, concha 16, helix 18, and tragus 20. The ear canal 22 includes a proximal section 21 between the tragus 20 and a first bend 24 of the canal 22. A middle section 27 is shown between the first bend 24 and a second bend 26 of the canal 22. A distal section 29 is shown between the second bend 26 and an ear drum 28.

Most ear-worn electronic devices, including ITE and ITC hearing aids, extend into the ear 10 and end short of the second bend 26. These devices cannot be made to extend further due to the discomfort and the difficulty of inserting the device in the canal 22 past the second bend 26. Extending the device structure into the proximity of or past the second bend 26 also increases movement of the ear-worn electronic device during jaw movement. This movement can cause interference with sensor measurements. Moreover, the distance from the end of the ITE/ITC hearing aids and the second bend 26 varies from person-to-person, as does the diameter of the ear canal 22. In addition, the acoustic transducer of the hearing aid is placed into the end of the ITE/ITC shell or housing, and the hearing aid is prone to damage from ear wax in this area.

Embodiments of the disclosure are directed to an ear-worn electronic device which includes a waveguide extension that extends beyond the shell or housing of the device. Embodiments are directed to a waveguide extension configured to extend beyond the second bend 26 of the ear canal 22 without causing discomfort to a wearer of the ear-worn electronic device. The waveguide extension is communicatively coupled to an inner waveguide disposed within the housing of the ear-worn electronic device. An articulation arrangement, such as a hinge or a spring clip arrangement, mechanically couples the waveguide extension to the inner waveguide. The articulation arrangement allows the waveguide extension to articulate relative to the inner waveguide, thereby facilitating insertion of the waveguide extension past the second bend 26 without causing pain to the wearer of the ear-worn electronic device.

According to some embodiments, a waveguide extension is configured to extend from the end of a conventional hearing aid housing in order to point precisely towards the tympanic membrane or an area of the inner ear canal. As discussed above, the waveguide extension includes a hinge or spring clip feature allowing the waveguide extension to be bent around the second bend 26 of the ear canal 22 for ease of insertion. In addition, the hinge or spring clip feature is configured to cause the waveguide extension to be held against the inner side 23 of the canal where jaw motion is minimized and off the outer canal wall 25 where jaw motion is maximal. The inner waveguide and waveguide extension can be used for directing any waveform from the tympanic membrane and intra-aural area including optical and/or acoustic waveforms.

The inner waveguide and waveguide extension can be used to guide blackbody infrared radiation from the ear or it can be used in conjunction with an engineered light source within the ear-worn electronic device. For example, the inner waveguide and waveguide extension can be configured to both transmit light from an LED and collect a reflected signal back from the ear canal. In another example, the inner waveguide and waveguide extension can be configured to communicate blackbody IR radiation from the tympanic membrane to an IR sensor within the ear-worn electronic device configured to measure core body temperature, such as for mapping the temperature gradient of the inner ear. The waveforms communicated along the inner waveguide and waveguide extension can be used for various physiological measurements and/or for detecting ear wax. The waveguide extension can also be used as a structure to hold a disposable (e.g., detachable) sleeve that getters ear wax to prevent ear wax from contaminating the components of the ear-worn electronic device.

According to some embodiments, the inner waveguide and the waveguide extension form a two-part waveguide that can be formed from IR opaque material and bent into shape during manufacturing. The shape imparted to the waveguide extension is retained during use within the ear canal. The inner waveguide is disposed within the housing of the ear-worn electronic device and extends from innermost hole that houses the acoustic transducer to any position that supports a sensor, such as an infrared (IR) sensor. The inner waveguide preferably has an inner diameter sufficient to encompass the sensing portion of the IR sensor. The end of the inner waveguide can be attached to or around the IR sensor with any adhesive. The waveguide extension is hingedly or pivotally attached to the device housing at a location where the waveguide extension contacts the inner curvature side of the second bend (the arch side of the second bend) so that the waveguide extension can be bent inwards when inserting it into the ear canal.

The waveguide extension has a diameter smaller than that of the ear canal and can be configured so that sound travels through it or around it. The distal portion of the waveguide extension is configured to extend past the second bend (but terminate prior to the tympanic membrane) and is aimed at the tympanic membrane or a specified area of the inner ear canal. The distal portion of the waveguide extension can be encased in a soft polymer material of sufficient width to prevent the waveguide extension from penetrating into the side of the canal.

FIG. 1B is an illustration of an ear-worn electronic device having an inner waveguide and a waveguide extension in accordance with various embodiments. The ear-worn electronic device 100 includes a shell or housing 102 having a shape that allows for insertion of the device 100 into the ear canal of a wearer of the device 100. According to some embodiments, the ear-worn electronic device 100 is configured as an ITE or ITC hearing aid. The housing 102 includes a proximal section comprising a proximal end 104 and a distal section comprising a distal end 106. When the ear-worn electronic device is positioned within the ear, the proximal end 104 is exposed to the outer ear and the environment, and the distal end 106 is positioned within the ear canal. It is understood that the distal end 106 is the terminal end of the housing 102 of the ear-worn electronic device 100.

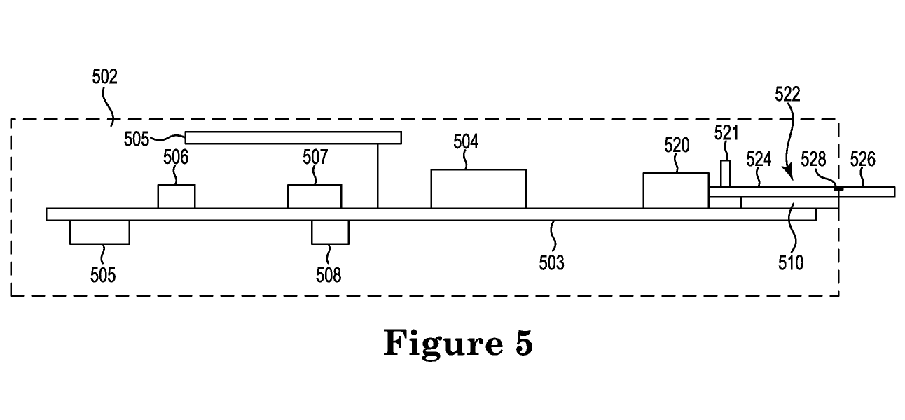

The housing 102 of the ear-worn electronic device 100 encompasses a number of components which are not shown in FIG. 1B. As will be described hereinbelow, FIG. 5 shows various components that are typically disposed within the housing 102. For purposes of clarity, FIG. 1B and other figures illustrate features of an inner waveguide and waveguide extension of the ear-worn electronic device 100 in accordance with various embodiments.

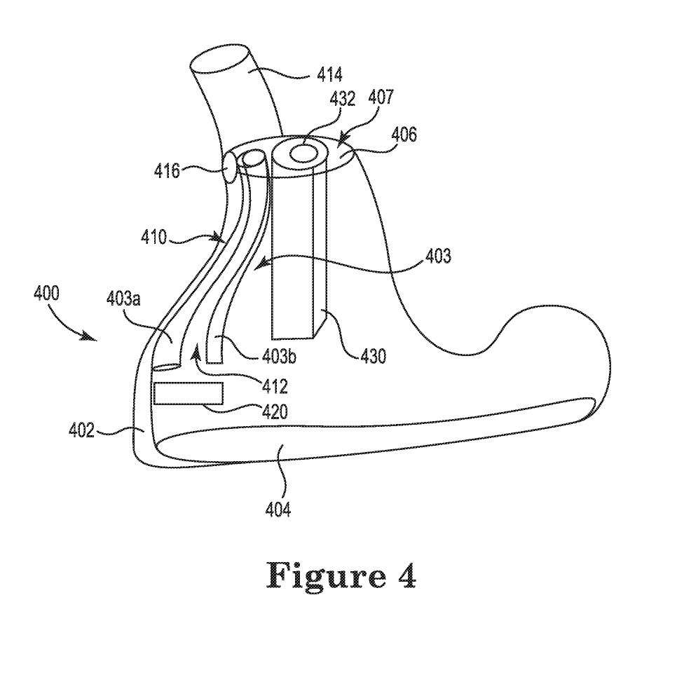

The ear-worn electronic device 100 includes a waveguide 110 which includes an inner waveguide 112 and a waveguide extension 114. According to various embodiments, the inner waveguide 112 comprises a flexible hollow tube that extend from an opening 107 at the distal end 106 of the housing 102 to a sensor 120 positioned within the housing 102. In other embodiments, the inner waveguide 112 comprises a void within a sidewall of the housing 102 (see, e.g., FIG. 4). The waveguide extension 114 comprises a flexible hollow tube that communicates with the inner waveguide 112 and extends outwardly from the opening 107 at the distal end 106 of the housing 102. An articulation arrangement 116 is connected to the inner waveguide 112 and the waveguide extension 114 or a portion of the housing 102 adjacent to the waveguide extension 114. The articulation mechanism 116 is configured to allow the waveguide extension 114 to articulate relative to the inner waveguide 112. The articulation arrangement can comprise a hinge or a spring clip, for example.

The inner surface of the inner waveguide 112 and the waveguide extension 114 is coated with or comprises a material that facilitates transmission of waveforms along the inner waveguide 112. In the case of infrared waveforms, for example, the inner surface of the inner waveguide 112 and the waveguide extension 114 is coated with an optically opaque material, such as an IR opaque material. Suitable materials include aluminum, silver, gold, or other materials opaque to infrared wavelengths in the region of 10 μm. In some embodiments, the inner waveguide 112 and waveguide extension 114 are flexible hollow tubes formed from an IR opaque material. In other embodiments, one or both of the inner waveguide 112 and waveguide extension 114 are formed from polymeric tubes having an inner surface coated with an IR opaque material. For example, a material layer can be formed on an inner surface of one or both of the inner waveguide 112 and the waveguide extension 114 using a material deposited by vapor or chemical deposition or other techniques. It is understood that, although the waveguide extension 114 comprises a flexible hollow tube, the inherent flexibility of the waveguide extension 114 is insufficient to accommodate the second bend of the ear canal without causing pain to the wearer of the device 100. It is noted that a waveguide extension 114 that is too flexible will not retain its desired pre-shape once inserted past the second bend. Also, a waveguide extension 114 that is too flexible would undesirably contact the outer canal wall where jaw motion is maximal.

As shown, the sensor 120 is mounted to or supported by the proximal end 104 of the housing 102. The sensor 120 is typically mounted to a substrate, which may be flexible or rigid, within the housing 102 and supported by a spine structure of the housing 102. The spine can be supported by the proximal end 104 and/or the sidewalls of the housing 102. The sensor 120 is positioned relative to the inner waveguide 112 so that waveforms communicated along the inner waveguide 112 impinge on the sensing element of the sensor 120. The sensor 120 can be a passive sensor (sensing only, no emitter). The sensor 120 can be an active sensor (emitter plus sensing), comprising both a sensor element and a source element (e.g., a light source, such as an LED, or an acoustic source). In some embodiments, the sensor 120 is an optical sensor, such as an infrared (IR) sensor, configured for sensing core body temperature. For example, the sensor 120 can be configured for sensing blackbody infrared radiation from the ear. In other embodiments the sensor 120 is an acoustic sensor. In further embodiments, the sensor 120 is a combination of an IR sensor and an acoustic sensor.

According to some embodiments, the location and contour of the inner waveguide 112 within the housing 102 can vary in three manners based on the individual ear geometry and the custom placement of the electronic components within the housing 102. First, the inner waveguide 112 needs to be aligned to the sensor 120 mounted within the housing 102. Second, the distal end 106 of the inner waveguide 112 needs to be optimally positioned relative to the distal opening 106 in order to access the ear canal. Third, the mid-section of the inner waveguide 112 may need to be bent around the internal components of the housing 102. The inner waveguide 112 can be implemented to address each of these three considerations.

As discussed previously, the hinge or spring clip 116 is connected to the inner waveguide 112 and the waveguide extension 114 or a portion of the housing 102 adjacent to the waveguide extension 114, and allows the waveguide extension 114 to articulate relative to the inner waveguide 112. The hinge or spring clip 116 is situated on the waveguide 110 so that is positioned adjacent to the inner curvature side of the second bend of the ear canal. At this location, the waveguide extension 114 can articulate inwardly when the waveguide extension 114 is inserted into the ear canal.

The hinge or spring clip 116 can include a spring mechanism that biases a contacting surface of the waveguide extension 114 into engagement with a contacting surface of the inner waveguide 112. During insertion of the ear-worn electronic device 100 into the ear canal, a region of the waveguide extension 114 opposing the hinge or spring clip 116 pivots away from the inner waveguide 112 so that the waveguide extension 114 can bend around the second bend of the ear canal during insertion. After the waveguide extension 114 is positioned past the second bend, the waveguide extension 114 pivots back towards and re-engages with the inner waveguide 112. In a fully inserted configuration within the ear canal, a continuous waveguide is defined between the inner waveguide 112 and the waveguide extension 114.

According to some embodiments, the hinge or spring clip 116 is configured to prevent the waveguide extension 114 from contacting the ear canal on the side of the second bend where maximum expansion occurs during jaw motion. To further prevent unwanted contact between the waveguide extension 114 and the side of the ear canal that experiences maximum expansion during jaw motion, the diameter of the waveguide extension 114 is made smaller than the diameter of the portion of the ear canal between the second bend and the eardrum. In some embodiments, the hinge or spring clip 116 can be implemented such that the waveguide 110 can be purposely bent at the joint between the inner waveguide 112 and the waveguide extension 114 for insertion of the device 100 into the ear. The hinge or spring clip 116 can exert a force at the joint which can be overcome by an insertion force in excess of a force produced by jaw movement.

The hinge or spring clip 116 can be implemented such that the waveguide extension 114 will oscillate at a frequency outside the frequency range of interest for any selected movement (e.g., jaw motion during eating) during a measurement taken at the tympanic membrane or the inner ear canal. For example, the hinge or spring clip 116 and waveguide extension 114 can be designed to eliminate, reduce or control motion in order to eliminate motion artifacts for a heart rate monitoring measurement. This is particularly relevant for jaw motion during talking or chewing which often occurs at the same frequency as the heart rate and cannot be removed using signal processing. Any resulting movement artifacts can be separated out mathematically by an equation, such as a Fourier transform that converts a time domain signal into a frequency domain.

The waveguide 110 can be used in conjunction with the optical sensor 120 and a light source or light sources and associated electronic components to detect a variety of physiologic signals or conditions from the tympanic membrane or the inner ear canal. Representative physiologic signals or conditions include core body temperature, heart rate, heart rate variability, and oxygen saturation. In embodiments that utilize a passive optical sensor 120 (no light source), for example, the waveguide 110 can be used in conjunction with the optical sensor 120 and its associated electronic components to detect heart rate and heart rate variability from the innate infrared radiation of the body at the tympanic membrane. The waveguide 110 can also be used in conjunction with a microphone to transmit acoustical waveforms from the ear for purposes of detecting a heart rate, such as in the manner disclosed in US Published Application No. 2014/0288453, which is incorporated herein by reference. Because physiologic measurements are made within the ear canal and well away from the outer ear, ambient light artifacts are significantly reduced or minimized.

The end of the waveguide extension 114 is aimed at the tympanic membrane or a specified area of the innermost ear canal in order to increase the accuracy of measuring any signal during exposure to cold. Sympathetic mediated vasoconstriction from cold exposure occurs more predominately in the outer areas of the ear causing diminished signal intensity and flattened peak geometry and subsequent lack of measurement accuracy. Because physiologic measurements are made within the ear canal and well away from the outer ear, the effects of sympathetic mediated vasoconstriction from cold exposure are significantly reduced or eliminated.

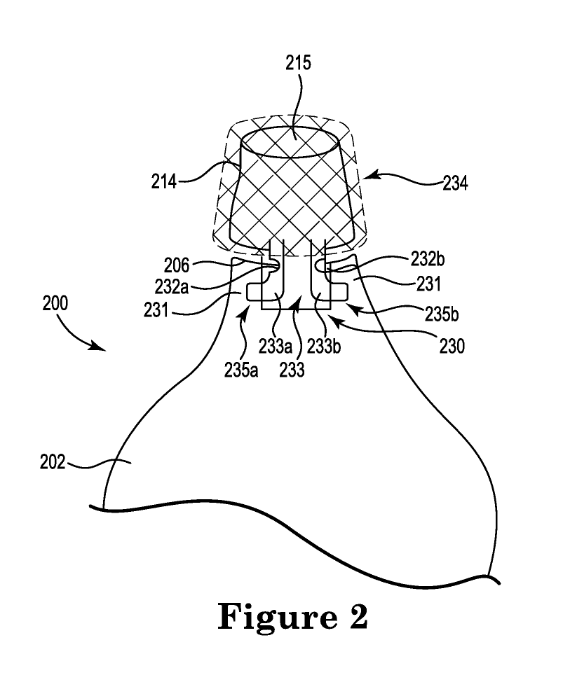

FIG. 2 is an illustration of an ear-worn electronic device having a detachable waveguide extension in accordance with various embodiments. The ear-worn electronic device 200 shown in FIG. 2 includes a housing 202 and components (e.g., an inner waveguide, electronics, etc.) the same as or similar to those of the embodiment shown in FIG. 1B. For purposes of clarity, the inner waveguide, IR sensor, and other components of the ear-worn electronic device 200 are not shown in FIG. 2.

FIG. 2 shows details of a detachable waveguide extension 214 installed at the distal opening 206 of the housing 202. The waveguide extension 214 incorporates a spring clip 233 having a first leg 233a and a second leg 233b. The spring clip 233 is configured to maintain engagement between the waveguide extension 214 and the housing 202 while allowing the waveguide extension 214 to articulate relative to the housing 202. More particularly, the spring clip 233 is configured to allow the waveguide extension 214 to bend inwardly at the inner curvature side of the second bend of the ear canal when the housing 202 is inserted into the ear canal.

The first and second legs 233a and 233b respectively include a depression 232a and 232b configured to receive a pinching tool, such as tweezers. Application of a pinching force at the depressions 232a and 232b causes inward deflection of the first and second legs 233a and 233b, allowing the spring clip 233 to be retentively installed within the distal opening 206 of the housing 202. The distal wall 231 of the housing 202 proximate the distal opening 206 includes recesses 235a and 235b dimensioned to receive outward projecting tabs of the first and second legs 233a and 233b. When the first and second legs 233a and 233b reach the recesses 235a and 235b, the outwardly projecting tabs of the first and second legs 233a and 233b move into the recesses 235a and 235b in response to a spring force generated by the spring clip 233. In this installed configuration, the waveguide extension 214 matingly engages with the inner waveguide, allowing for communication of waveforms between the distal opening 215 of the waveguide extension 214 and an IR and/or acoustic sensor disposed in the housing 202. It is noted that, in some embodiments, the detachable waveguide extension 214 is a disposable (and replaceable) component of the ear-worn electronic device 200.

According to some embodiments, all or a portion of the waveguide extension 214 can be covered by a mesh sleeve 234 configured to getter or harvest ear wax. In general, the mesh sleeve 234 is configured to preferentially getter ear wax compared to the housing 202 or the acoustic transducer and transducer housing. The mesh sleeve 234 can be manufactured as a disposable component that can be installed on and removed from the waveguide extension 214 based on the magnitude of ear wax buildup on the sleeve 234. In some embodiments, the waveguide extension 214 and the mesh sleeve 234 are configured as disposable components of the ear-worn electronic device 200. The mesh sleeve 234 can be manufactured as a sterile component. As was discussed previously, waveforms communicated along the inner waveguide and waveguide extension 214 can be used for detecting ear wax. For example, an optical sensor and a light source can be used to detect the level of ear wax buildup on the mesh sleeve 234. In response to the level of ear wax exceeding a threshold, an indicator (e.g., an LED or audible sound) of the ear-worn electronic device 200 can inform the wearer that the mesh sleeve 234 should be replaced with a new mesh sleeve 234.

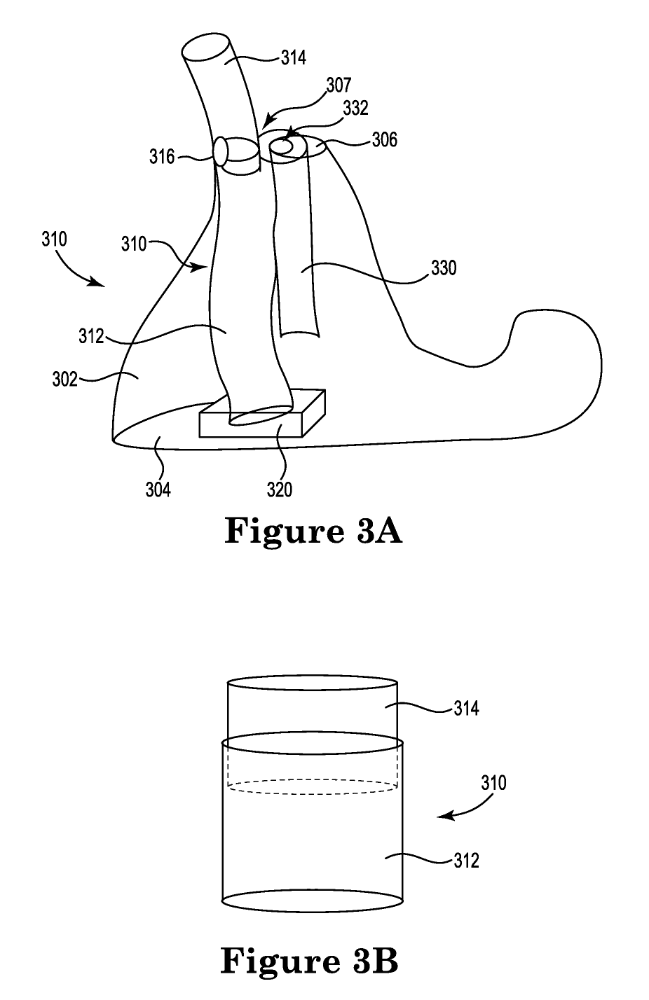

FIG. 3A illustrates an ear-worn electronic device having an inner waveguide and a waveguide extension in accordance with various embodiments. FIG. 3B shows details of the inner waveguide and the waveguide extension illustrated in FIG. 3A. The ear-worn electronic device 300 shown in FIG. 3A includes a housing 302 and components (e.g., electronics, etc.) the same as or similar to those of the embodiment shown in FIG. 1B.

The housing 302 includes a proximal end 304 and a distal end 306 having an opening 307. The housing 302 is configured to support a two-part waveguide 310 comprising an inner waveguide 312 and a waveguide extension 314. A hinge or spring clip 316 is connected to the inner waveguide 312 and the waveguide extension 314 or a portion of the housing 302 adjacent to the waveguide extension 314, and operates in a manner previously described. Disposed within the housing 302 is the inner waveguide 312 communicatively coupled to a sensor 320 shown mounted to or supported by the proximal end 304. In this embodiment, the sensor 320 is an IR sensor, which is arranged so that a sensing element of the IR sensor 320 is encompassed by the inner waveguide 312. The waveguide extension 314 extends outwardly from the distal end 306 of the housing 302 and is communicatively coupled to the inner waveguide 312. In the embodiment shown in FIG. 3A, and as best seen in FIG. 3B, the waveguide extension 314 is dimensioned to fit within the inner waveguide 312 (e.g., in a telescoping manner). More particularly, the diameter of the waveguide extension 314 is smaller than that of the inner waveguide 312.

In the embodiment shown in FIG. 3A, an acoustic transducer 330 is positioned alongside the inner waveguide 312. The acoustic transducer 330 is configured to convert electrical signals to acoustic signals, and to communicate the acoustic signals into the ear canal. As shown, an output 332 of the acoustic transducer 330 has access to the ear canal via the opening 307 at the distal end 306 of the housing 302. In the embodiment shown in FIG. 3A, the opening 307 at the distal end 306 accommodates the waveguide extension 314 and the output 332 of the acoustic transducer 330. In some embodiments, the output 332 of the acoustic transducer 306 is coupled to the waveguide 310 such that acoustic waveforms are transmitted to/from the ear canal via the waveguide 310. In further embodiments, the output 332 of the acoustic transducer 306 is coupled to the waveguide 310 such that both optical and acoustic waveforms are communicated along the waveguide 310.

FIG. 4 illustrates an ear-worn electronic device having an inner waveguide and a waveguide extension in accordance with various embodiments. The ear-worn electronic device 400 shown in FIG. 4 includes a housing 402 and components (e.g., electronics, etc.) the same as or similar to those of the embodiment shown in FIG. 1B. The housing 402 includes a proximal end 404 and a distal end 406 having an opening 407. The housing 402 is configured to support a two-part waveguide 410 comprising an inner waveguide 412 and a waveguide extension 414. A hinge or spring clip 416 is connected to the inner waveguide 412 and the housing 402 adjacent to the waveguide extension 414 and operates in a manner previously described.

Disposed within the housing 402 is the inner waveguide 412 communicatively coupled to a sensor 420 shown mounted to or supported by the proximal and 404. In this embodiment, the sensor 420 is an IR sensor, which is arranged so that a sensing element of the IR sensor 420 is encompassed by the inner waveguide 412. The waveguide extension 414 extends outwardly from the distal end 406 of the housing 402 and is communicatively coupled to the inner waveguide 412. As shown, an acoustic transducer 430 is positioned alongside the inner waveguide 412, and includes an output 432 positioned at the opening 407 of the housing's distal and 406.

In the embodiment shown in FIG. 4, the inner waveguide 412 is fabricated into a sidewall 403 of the housing 402. In this embodiment, the inner waveguide 412 is a void defined between an inner wall portion 403b and an outer wall portion 403a of the sidewall 403. According to various embodiments, an interior wall of the inner waveguide 412 includes a metallization layer. The metallization layer can be deposited via a chemical or vapor deposition process. In some embodiments, a metal tube can be inserted into the void within the sidewall 403. The metallization layer or metal tube can be formed from an IR opaque material, such as aluminum, silver, gold, or other material opaque to infrared wavelengths in the region of 10 μm.

FIG. 5 is a block diagram showing various components of an ear-worn electronic device 502 that can be configured to incorporate a waveguide comprising an inner waveguide and a waveguide extension in accordance with various embodiments. The block diagram of FIG. 5 shows components of an ear-worn electronic device 502 that can be implemented in accordance with the embodiments shown in FIGS. 1-4. It is understood that an ear-worn electronic device 502 may exclude some of the components shown in FIG. 5 and/or include additional components. It is also understood that the ear-worn electronic device 502 illustrated in FIG. 5 can be either a right ear-worn device or a left-ear worn device. The components of the right and left ear-worn devices can be the same or different.

The ear-worn electronic device 502 shown in FIG. 5 includes several components electrically connected to a mother flexible circuit 503. A battery 505 is electrically connected to the mother flexible circuit 503 and provides power to the various components of the ear-worn electronic device 502. One or more microphones 506 are electrically connected to the mother flexible circuit 503, which provides electrical communication between the microphones 506 and a DSP (digital signal processor) 504. Among other components, the DSP 504 can incorporate or be coupled to audio signal processing circuitry and optical sensor processing circuitry. One or more user switches 508 (e.g., on/off, volume, mic directional settings, mode selection) are electrically coupled to the DSP 504 via the flexible mother circuit 503.

A sensor 520 is coupled to the DSP 504, such as via the mother flexible circuit 503. The sensor 520 can be an IR sensor of a type previously described. The ear-worn electronic device 502 includes a two-part waveguide 522 comprising an inner waveguide 524 and a waveguide extension 526 that extends from a housing of the device 502. A hinge or spring clip 528 (or other articulation mechanism) is connected to the waveguide extension 526 and the inner waveguide 524 or a portion of the device housing adjacent to the inner waveguide 524. The hinge or spring clip 528 operates in a manner previously described. The inner waveguide 524 is communicatively coupled to a sensing element of the sensor 520. In some embodiments, one or more light sources 521 can be coupled to the inner waveguide 524. The DSP 504, in cooperation with the sensor 520, light source(s) 521, and waveguide 522, can be configured to measure a variety of physiologic signals or conditions (e.g., core body temperature, heart rate, heart rate variability, and oxygen saturation) using waveforms developed or derived from the tympanic membrane or a specified area of the inner ear canal.

An audio output device 510, such as an acoustic transducer, is electrically connected to the DSP 504 via the flexible mother circuit 503. The audio output device 510 comprises a speaker (coupled to an amplifier). The ear-worn electronic device 502 may incorporate a communication device 507 coupled to the flexible mother circuit 503 and to an antenna 505 directly or indirectly via the flexible mother circuit 503. The communication device 507 can be a Bluetooth® transceiver, such as a BLE (Bluetooth® low energy) transceiver or other transceiver (e.g., an IEEE 802.11 compliant device). The communication device 507 can be configured to communicate with an external device, such as a smartphone or laptop, in accordance with various embodiments.

The embodiments discussed hereinabove are generally directed to an ear-worn electronic device, such as an ITE or ITC hearing aid. In some embodiments, a waveguide extension and hinge/spring clip feature of a type described hereinabove can be configured for use on a standard disposable in-the-ear end piece for a clinical IR temperature thermometer. The placement of the tip of the thermometer is critical to the measurement and highly non-repeatable due to the second bend obstruction. A waveguide extension and hinge/spring clip feature of the present disclosure can be added as components of a disposable in-the-ear end piece for a clinical IR temperature thermometer. It is noted that, in a limited number of anatomical cases, the inner waveguide and waveguide extension can be one continuous piece that extends past the second bend. The waveguide extension can serve the dual purpose of protecting the ear-worn electronic device (e.g., ITE or ITC hearing aid) from ear wax.

This document discloses numerous embodiments, including but not limited to the following:

Item 1 is an ear-worn electronic device, comprising:

a housing configured for insertion into an ear canal and comprising a proximal section and a distal section, the distal section comprising a distal end configured to terminate prior to a second bend of the ear canal when the housing is fully inserted into the ear canal;

an inner waveguide disposed in the housing and extending to the distal end;

a waveguide extension coupled to the distal end and dimensioned to extend past the second bend of the ear canal when the housing is fully inserted into the ear canal, the waveguide extension communicatively coupled to the inner waveguide; and

an articulation mechanism between the waveguide and the waveguide extension, the articulation mechanism configured to facilitate articulation of the waveguide extension relative to the inner waveguide during insertion of the housing into the ear canal.

Item 2 is the device of item 1, wherein the inner waveguide and the waveguide extension each comprise an inner surface formed from or coated with an infrared-opaque material.
Item 3 is the device of item 1, wherein the inner waveguide and the waveguide extension each comprise a tube formed from an infrared-opaque material.
Item 4 is the device of item 1, wherein the inner waveguide is disposed within a sidewall of the housing.
Item 5 is the device of item 4, wherein an inner surface of the inner waveguide is coated with an infrared-opaque material.
Item 6 is the device of item 1, wherein the articulation mechanism comprises a hinge or a spring clip.
Item 7 is the device of item 1, wherein the articulation mechanism is situated on the housing such that the waveguide extension contacts an arch side of the second bend when the housing is fully inserted into the ear canal.
Item 8 is the device of item 1, wherein the articulation mechanism is configured to bias the waveguide extension against a low movement side of the ear canal.
Item 9 is the device of item 1, wherein the waveguide extension is directed toward a tympanic membrane or a specified area of the ear canal when the housing is fully inserted into the ear canal.
Item 10 is the device of item 1, wherein the waveguide extension comprises a mesh sleeve configured to getter ear wax.
Item 11 is the device of item 10, wherein the mesh sleeve is detachable from the waveguide extension.
Item 12 is the device of item 1, wherein:

the waveguide extension has a diameter smaller than that of the inner waveguide; and

at least a portion of the waveguide extension is disposed within the inner waveguide.

Item 13 is the device of item 1, further comprising an infrared sensor communicatively coupled to the inner waveguide.
Item 14 is the device of item 1, further comprising an infrared sensor and a light source communicatively coupled to the inner waveguide.
Item 15 is the device of item 1, further comprising an acoustic transducer proximate the inner waveguide.
Item 16 is the device of item 1, wherein the inner waveguide and the waveguide extension are configured to facilitate communication of optical waveforms.
Item 17 is the device of item 1, wherein the inner waveguide and the waveguide extension are configured to facilitate communication of acoustic waveforms.
Item 18 is the device of item 1, wherein the inner waveguide and the waveguide extension are configured to facilitate communication of optical and acoustic waveforms.
Item 19 is an ear-worn electronic device, comprising:

a housing configured for insertion into an ear canal and comprising a proximal section and a distal section, the distal section comprising a distal end configured to terminate prior to a second bend of the ear canal when the housing is fully inserted into the ear canal;

an inner waveguide disposed in the housing and extending to the distal end;

a waveguide extension coupled to the distal end and dimensioned to extend past the second bend of the ear canal when the housing is fully inserted into the ear canal, the waveguide extension communicatively coupled to the inner waveguide;

an articulation mechanism between the waveguide and the waveguide extension, the articulation mechanism configured to facilitate articulation of the waveguide extension relative to the inner waveguide during insertion of the housing into the ear canal;

an infrared sensor disposed in the housing and communicatively coupled to the inner waveguide; and

a processor disposed in the housing and coupled to the infrared sensor, the processor configured to measure a physiologic signal or condition in response to a waveform received by the infrared sensor.

Item 20 is the device of item 19, wherein the physiologic signal or condition comprises core body temperature.
Item 21 is the device of item 19, wherein the physiologic signal or condition comprises a cardiac signal or condition.
Item 22 is the device of item 19, wherein the physiologic signal or condition comprises heart rate, heart rate variability, or oxygen saturation.
Item 23 is the device of item 19, wherein the inner waveguide and the waveguide extension each comprise an inner surface formed from or coated with an infrared-opaque material.
Item 24 is the device of item 19, wherein the inner waveguide and the waveguide extension each comprise a tube formed from an infrared-opaque material.
Item 25 is the device of item 19, wherein the inner waveguide is disposed within a sidewall of the housing.
Item 26 is the device of item 25, wherein an inner surface of the inner waveguide is coated with an infrared-opaque material.
Item 27 is the device of item 19, wherein the articulation mechanism comprises a hinge or a spring clip.
Item 28 is the device of item 19, wherein the articulation mechanism is situated on the housing such that the waveguide extension contacts an arch side of the second bend when the housing is fully inserted into the ear canal.
Item 29 is the device of item 19, wherein the articulation mechanism is configured to bias the waveguide extension against a low movement side of the ear canal.
Item 30 is the device of item 19, wherein the waveguide extension is directed toward a tympanic membrane or a specified area of the ear canal when the housing is fully inserted into the ear canal.
Item 31 is the device of item 19, wherein the waveguide extension comprises a mesh sleeve configured to getter ear wax.
Item 32 is the device of item 31, wherein the mesh sleeve is detachable from the waveguide extension.
Item 33 is the device of item 19, wherein:

the waveguide extension has a diameter smaller than that of the inner waveguide; and

at least a portion of the waveguide extension is disposed within the inner waveguide.

Item 34 is the device of item 19, further a light source communicatively coupled to the inner waveguide.
Item 35 is the device of item 19, further comprising an acoustic transducer proximate the inner waveguide.
Item 36 is the device of item 19, wherein the inner waveguide and the waveguide extension are configured to facilitate communication of optical waveforms.
Item 37 is the device of item 19, wherein the inner waveguide and the waveguide extension are configured to facilitate communication of acoustic waveforms.
Item 38 is the device of item 19, wherein the inner waveguide and the waveguide extension are configured to facilitate communication of optical and acoustic waveforms.

Although the subject matter has been described in language specific to structural features and/or methodological acts, it is to be understood that the subject matter defined in the appended claims is not necessarily limited to the specific features or acts described above. Rather, the specific features and acts described above are disclosed as representative forms of implementing the claims.

What is claimed is:

1. An ear-worn electronic device, comprising:
a housing configured for insertion into an ear canal and comprising a proximal section and a distal section, the distal section comprising a distal end configured to terminate prior to a second bend of the ear canal when the housing is fully inserted into the ear canal;
an inner waveguide disposed in the housing and extending to the distal end;
a waveguide extension coupled to the distal end and dimensioned to extend past the second bend of the ear canal when the housing is fully inserted into the ear canal, the waveguide extension communicatively coupled to the inner waveguide; and
an articulation mechanism between the waveguide and the waveguide extension, the articulation mechanism configured to facilitate articulation of the waveguide extension relative to the inner waveguide during insertion of the housing into the ear canal.

2. The device of claim 1, wherein the inner waveguide and the waveguide extension each comprise an inner surface comprising an infrared-opaque material.

3. The device of claim 1, wherein the inner waveguide and the waveguide extension each comprise a tube comprising an infrared-opaque material.

4. The device of claim 1, wherein the inner waveguide is disposed within a sidewall of the housing.

5. The device of claim 1, wherein the articulation mechanism comprises a hinge or a spring clip.

6. The device of claim 1, wherein the articulation mechanism is situated on the housing such that the waveguide extension contacts an arch side of the second bend when the housing is fully inserted into the ear canal.

7. The device of claim 1, wherein the waveguide extension is directed toward a tympanic membrane or a specified area of the ear canal when the housing is fully inserted into the ear canal.

8. The device of claim 1, wherein:
the waveguide extension comprises a mesh sleeve configured to getter ear wax; and
the mesh sleeve is detachable from the waveguide extension.

9. The device of claim 1, further comprising an infrared sensor communicatively coupled to the inner waveguide.

10. The device of claim 1, further comprising an infrared sensor and a light source communicatively coupled to the inner waveguide.

11. The device of claim 1, further comprising an acoustic transducer proximate the inner waveguide.

12. An ear-worn electronic device, comprising:
a housing configured for insertion into an ear canal and comprising a proximal section and a distal section, the distal section comprising a distal end configured to terminate prior to a second bend of the ear canal when the housing is fully inserted into the ear canal;
an inner waveguide disposed in the housing and extending to the distal end;
a waveguide extension coupled to the distal end and dimensioned to extend past the second bend of the ear canal when the housing is fully inserted into the ear canal, the waveguide extension communicatively coupled to the inner waveguide;
an articulation mechanism between the waveguide and the waveguide extension, the articulation mechanism configured to facilitate articulation of the waveguide extension relative to the inner waveguide during insertion of the housing into the ear canal;
an infrared sensor disposed in the housing and communicatively coupled to the inner waveguide; and
a processor disposed in the housing and coupled to the infrared sensor, the processor configured to measure a physiologic signal or condition in response to a waveform received by the infrared sensor.

13. The device of claim 12, wherein the waveguide extension is directed toward a tympanic membrane or a specified area of the ear canal when the housing is fully inserted into the ear canal.

14. The device of claim 12, wherein the physiologic signal or condition comprises one or more of core body temperature, heart rate, heart rate variability, and oxygen saturation.

15. The device of claim 12, wherein the inner waveguide and the waveguide extension each comprise an inner surface formed from or coated with an infrared-opaque material.

16. The device of claim 12, wherein the inner waveguide is disposed within a sidewall of the housing.

17. The device of claim 12, wherein:
the articulation mechanism comprises a hinge or a spring clip; and
the articulation mechanism is situated on the housing such that the waveguide extension contacts an arch side of the second bend when the housing is fully inserted into the ear canal.

18. The device of claim 12, wherein:
the waveguide extension comprises a mesh sleeve configured to getter ear wax; and
the mesh sleeve is detachable from the waveguide extension.

19. The device of claim 12, further comprising a light source communicatively coupled to the inner waveguide.

20. The device of claim 12, further comprising an acoustic transducer proximate the inner waveguide.
